(19) United States
(12) Patent Application Publication
(10) Pub. No.: US 2008/0015479 A1
(43) Pub. Date: Jan. 17, 2008

(54) SLING DESIGN

(76) Inventor: Susan Soscia, Cranston, RI (US)

Correspondence Address: BARLOW, JOSEPHS & HOLMES, LTD., 101 DYER STREET, 5TH FLOOR, PROVIDENCE, RI, 02903, US

(21) Appl. No.: 11/773,653

(22) Filed: Jul. 5, 2007

Related U.S. Application Data
(60) Provisional application No. 60/806,949, filed on Jul. 11, 2006.

(51) Int. Cl. A61F 5/40

(52) U.S. Cl. 602/4

(57) ABSTRACT

A sling construction is provided that includes an improved support strap for more uniformly distributing the weight of the supported arm of the wearer's shoulder while preventing strap bunching and creep. The strap is preferably formed from an elastomer or a rubber that is relatively compliant yet has a low coefficient of elongation. This allows the strap to be highly supportive of the sling while also conforming to the contour of the wearer's neck and shoulders in a manner that makes the strap and sling comfortable to wear. The inner contact surface of the strap includes nubs or raised features that prevent the strap from sliding. Further, the improved strap support construction may be used in a modular fashion in conjunction with standard sling pouches or with an enhanced sling pouch in order to greatly improve the performance and appearance of the sling.

### CROSS-REFERENCE TO RELATED APPLICATIONS

This application is related to and claims priority from earlier filed U.S. Provisional Patent Application No. 60/806,949, filed Jul. 11, 2006, the contents of which are incorporated herein by reference.

### BACKGROUND OF THE INVENTION

The present invention relates generally to a sling assembly for comfortably supporting the arm of a wearer. More specifically, the present invention is directed to an improved sling assembly that provides enhanced ability to uniformly distribute the weight of the supported arm across the wearer's shoulder while also providing auxiliary storage space such that the combination tends to increase user compliance with the need to wear a sling.

In the medical and rehabilitative therapy industries, there commonly arises the need to support and/or immobilize the arm of a person against the torso of the body as treatment for an injury to various parts of the arm and/or shoulder. In particular, an injury to the shoulder presents difficulty because the rotational capabilities of the shoulder, while enhancing the function of the joint, also complicate treatment of the shoulder following injury or surgery. Such treatment frequently requires determining a desired optimal healing position of the shoulder and associated arm, placement of the shoulder and associated arm in the desired healing position and immobilization of the shoulder and arm in the desired healing position. This type of treatment is particularly applicable, but not limited, to soft tissue injuries involving damage to one or more connective shoulder ligaments and furthermore is oftentimes the treatment of choice following any number of surgical procedures, including surgery for recurrent posterior subluxation, rotator cuff surgery, humeral head or shaft fracture correction and the like. Healing occurs inter alia through diminution of inflammation and/or regeneration of muscle tissues, which is promoted by removing stress from the injured or surgically corrected joint. In most cases, such positioning of the shoulder and/or arm typically requires that the arm that extends from the injured shoulder be placed into an orthopedic support device so that the arm is supported while allowing the shoulder to heal.

Support devices, such as orthopedic braces, rigid casts, slings and the like are commonly employed alone or in combination for the positioning, support and immobilization of the shoulder with varying degrees of success. For example, rigid casts, which are typically molded plaster or resin, have traditionally been used as joint immobilizers. The rigid cast may be replaced from time to time as swelling is reduced. However, the disadvantages of rigid casts are well known. Rigid casts are heavy and uncomfortable to wear and are relatively laborious and complex to apply. Rigid casts may also unduly limit the mobility of the patient and cause joint stiffening and muscle atrophy. In addition, wound and skin treatments and bathing must usually be postponed until the rigid cast is removed.

Similarly, there are numerous problems associated with conventional arm slings or supports. These problems arise because such devices support the weight of the arm using a strap that passes over the wearer's shoulder and rests against the base of the wearer's neck. These problems are generally the result of the manner in which such slings are constructed. In particular, a traditional sling normally includes at least one strap that wraps around the neck or over the shoulder for supporting the arm cuff and thereby the arm in the desired orientation. In this arrangement, most of the weight of the arm is borne by the person's neck and collarbone on the opposite side of the supported arm. This can become very tiring and very uncomfortable, especially if the weight of a cast is also involved. The fact that the weight is borne fully by the narrow strap means that the strap consequently supports the entire weight of the arm such that the strap concentrates this force on a very small area of the body. Further contributing to this problem is the fact that the straps are formed from a light weight webbing that slides around on the users shoulder and tends to bunch up further concentrating the point to which the load is transferred. As a result, traditional arm slings are often uncomfortable thereby causing neck pain and frictional abrasion for the wearer and, in some extreme cases, may cause injuries of their own at the support contact points.

In an attempt to overcome the drawbacks resulting from the bunching and sliding of the sling strap, many prior art devices employ a pad arrangement that is installed into the strap at the location where the strap crosses the wearer's neck and shoulders. In most cases, this type of arrangement includes foam padding of some sort that is stitched into a fabric pocket that in turn surrounds the strap or is inserted into the strap, with the strap being attached at both ends thereof. With long term wear however, these pads become smelly and dirty while also breaking down under the load and losing their consistency. In most cases, these pads over time deteriorate to the point that they also bunch much like the fabric straps described above. Accordingly, even the padded straps begin to introduce increased and uneven distribution of weight across the wearer's shoulder and neck causing the wearer to stop using the sling.

Finally, conventional slings or arm supports are unsightly as they are formed from a fabric that typically emulates the hospital environment having a medicinal appearance in which they are implemented and appear as drab green or blue fabric. All of these drawbacks together generally result in a low frequency of compliance on the part of the user in wearing the necessary sling for the proscribed period of time.

Accordingly, there is a need for a sling that has an improved construction for better distributing the supported weight over the wearer's neck and shoulders. Further there is a need for a sling that includes a support strap that provides a more uniform and positive distribution of the supported weight without slipping or bunching so as to reduce stress on the wearer's cervical spine. There is still a further need for a sling design that employs a strap that has a uniform cross section that prevents bunching while also having a soft feel in order achieve an improved distribution of the supported weight in a manner that assists in increasing wearer compliance with the wearing of the sling. Finally, there is a need for such a sling assembly that includes an improved appearance and integrated storage pockets thereby further enhancing the wearability of the sling.

### BRIEF SUMMARY OF THE INVENTION

In this regard, the present invention provides a sling construction that includes an improved support strap for more uniformly distributing the weight of the supported arm of the wearer's shoulder while preventing strap bunching and creep. Further, the present invention provides for the use of the improved strap support construction in conjunction with an enhanced sling pouch in order to greatly improve the performance and appearance of the sling.

In accordance with the present invention, the strap portion of the sling is preferably formed from a compliant material that has a soft feel against the wearer's neck and shoulders yet that has sufficient body to resist bunching across its cross section. In this manner the width of the strap remains consistent over the contact areas on the wearer, thereby providing an even distribution of the supported weight. The strap is preferably formed from an elastomer or a rubber that is relatively compliant yet has a low coefficient of elongation. This allows the strap to be highly supportive of the sling while also conforming to the contour of the wearer's neck and shoulders in a manner that makes the strap and sling comfortable to wear. Further, the inner contact surface of the strap includes nubs or raised features that prevent the strap from sliding. The integrated non-slip features prevent the strap from moving, thereby eliminating irritation of the wearer's skin and also serve to provide better balance of the supported weight against the wearer's shoulders.

The strap of the present invention may be made as an integrated component with a sling pouch attached permanently thereto or as a retrofit strap that can be implemented with existing sling pouches. Building the strap in this manner allows for several advantageous features. First, the strap can be modular allowing its use with existing sling pouches. Second, the strap can be made to be easily adjustable by fitting its ends with hook and loop fastener strips and passing them through rings provided on the sling pouch. Finally, the strap can be marketed with modular interchangeable sling pouches so that a wearer can select slings that mach their personal fashion tastes or to coordinate with the garments that the sling will be worn over.

The sling pouch of the present invention is formed much like sling pouches of the prior art. The pouch has sidewalls that extend upwardly wherein the sidewalls are also attached at the rear of the pouch wherein the wearer's arm is inserted in the pouch and is supported from the bottom as well as behind the elbow. The attachment points for the strap are provided at the top of the sidewalls both at the rear where the sidewalls are closed and at the front where the sidewalls are open and the user's hand would extend therefrom. The strap may be stitched directly into the sling pouch as was stated above or threaded through rings that are affixed to the sling pouch to allow adjustment of the strap length and the relative height of the sling on the wearer.

Accordingly, it is an object of the present invention to provide a sling that has an improved construction for better distributing the supported weight over the wearer's neck and shoulders. It is a further object of the present invention to provide a sling that includes a support strap that provides a more uniform and positive distribution of the supported weight without slipping or bunching. It is still a further object of the present invention to provide a sling design that employs a strap that has a uniform cross section that prevents bunching while also having a soft feel in order achieve an improved distribution of the supported weight in a manner that assists in increasing wearer compliance with the wearing of the sling. Finally, it is an object of the invention to provide a sling assembly that includes an improved appearance and integrated storage pockets thereby further enhancing the wearability of the sling.

These together with other objects of the invention, along with various features of novelty, which characterize the invention, are pointed out with particularity in the claims annexed hereto and forming a part of this disclosure. For a better understanding of the invention, its operating advantages and the specific objects attained by its uses, reference should be had to the accompanying drawings and descriptive matter in which there is illustrated a preferred embodiment of the invention.

### BRIEF DESCRIPTION OF THE DRAWINGS

In the drawings which illustrate the best mode presently contemplated for carrying out the present invention:

FIG. 1 is a front perspective view of a the sling and support strap of the present invention supporting the arm of a wearer;

FIG. 2 is a front perspective view of the sling and support strap of the present invention depicting an embodiment with an adjustable length strap;

FIG. 3 is a perspective view of a fixed length one-piece support strap as provided in the present invention;

FIG. 4 is a perspective view of an adjustable length three-piece support strap as provided in the present invention; and

FIG. 5 is a cross sectional view taken along the line 5-5 in FIG. 4.

### DETAILED DESCRIPTION OF THE INVENTION

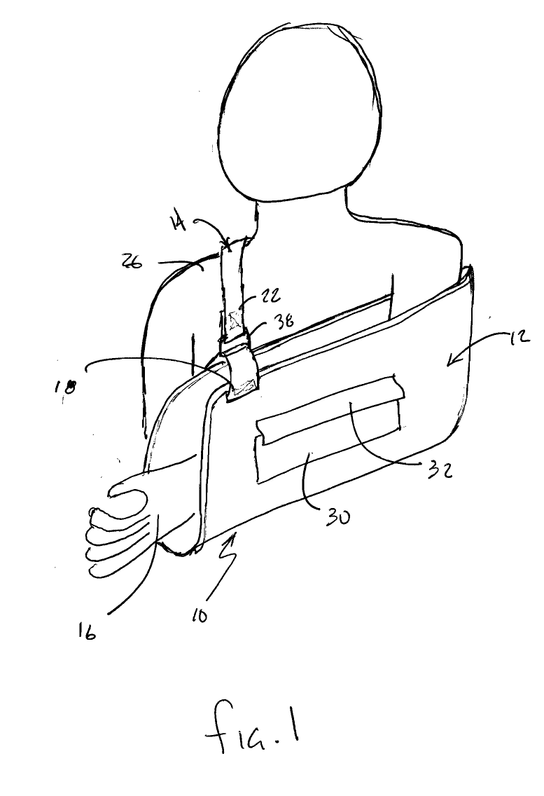

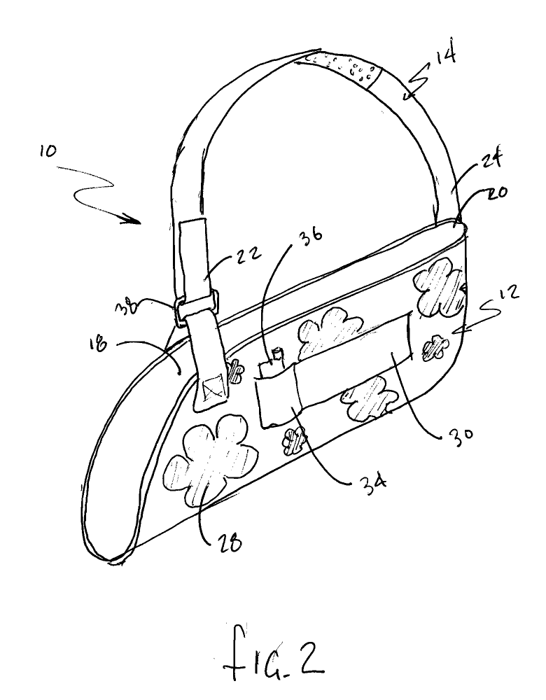

Now referring to the drawings, the sling assembly 10 including the sling pouch 12 and support strap 14 are shown and generally illustrated in FIGS. 1 and 2. The sling assembly 10 can be seen to generally include a sling pouch 12 and a sling support strap 14 that is affixed to the sling pouch 12. The sling pouch 12 is configured to receive the wearer's arm 16 in a substantially fixed position and includes first 18 and second 20 attachment points to which first 22 and second 24 ends of the support strap 14 are affixed. The support strap 14 extends upwardly from one of the attachment points 20 on the sling pouch 12, around the wearer's back, across their neck, over one of the wearer's shoulders 26 and back to the other attachment point 18 on the sling pouch 12. In this manner, the support strap 14 and sling pouch 12 cooperate to receive and support the wearer's arm 16 from a support surface on a wearer's body, such as for example the wearer's shoulder 26.

The sling pouch 12 is preferably a fabric pouch that cradles the wearer's arm 16 by extending upwardly along the sides thereof. Preferably, the back of the sling pouch 12 is closed so that the closed end serves to cradle the wearer's elbow. It is also preferable that the sling pouch 12 of the present invention be formed so as to be modular. In other words, the sling pouch 12 is formed so that the support strap 14 can easily be removed from the sling pouch 12 and a different sling pouch 12 substituted for use in the sling assembly 10. In this regard, the present invention anticipates that the sling pouches 12 can be produced in a variety of different fabrics and may include printed patterns 28. By offering different sling pouch 12 styles, the wearer is provided a broad range of sling pouch 12 selections. This allows a wearer to select a sling pouch 12 that most closely matches their fashion tastes or their particular wardrobe.

It can also be seen that the sling pouch 12 is formed to include a small pocket 30 on the front sidewall thereof that further includes a closure means in the form of a flap 32 that folds down over the pocket 30 opening. Similarly, this closure may be accomplished through the use of hook and loop fasteners, zippers, buttons, snaps or any other pocket closure means known to one skilled in the relevant art. The inclusion of the pocket 30 on the sling pouch 12 allows a wearer to store personal belongings such as keys, money, credit cards or other small items of value making the sling assembly 10 a convenience article and often eliminating the need for a purse as the wearing of a sling often conflicts with the carrying of a purse. As can best be seen in FIG. 2, the sling pouch 12 also preferably includes a second pocket 34 that is sized and particularly configured to receive and retain a cellular telephone or personal digital assistant (PDA) 36 to further enhance the convenience of the sling and reduce the need to carry other items in separate bags or the like.

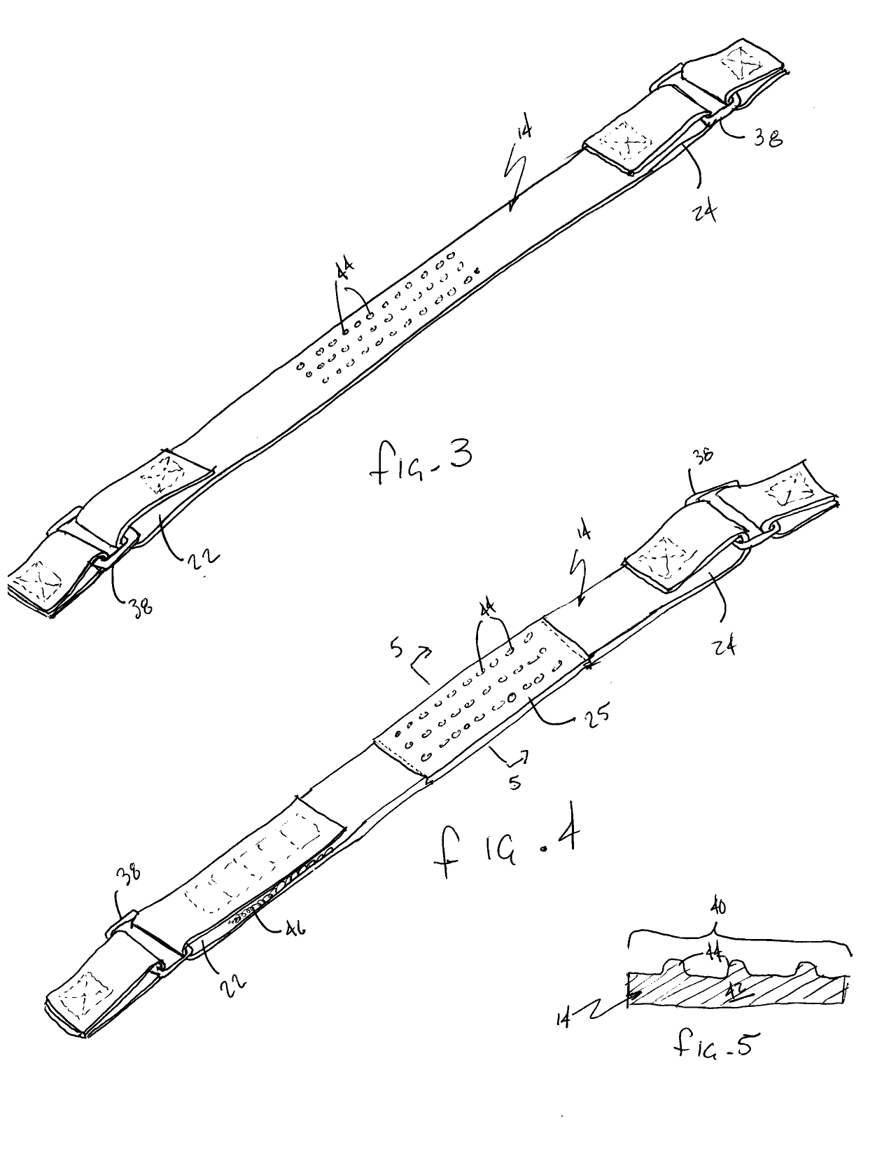

Referring again to FIG. 2, the support strap 14 can be seen to engage at a first end 22 thereof with a first attachment point 18 on the sling pouch 12 and at a second end thereof 24 with a second attachment point 20 on the sling pouch 12. Additionally, as can be seen in more detail in FIGS. 3 and 4, the support strap 14 may have a fixed length (FIG. 3) or an adjustable length (FIG. 4). Further the support strap 14 may be fixed on both ends, adjustable on both ends or have one fixed end and one adjustable end. The sling strap 14 may be affixed directly to the sling pouch 12 or may include D-rings 38 or other attachment loops that facilitate the sling pouch 12 and support strap 14 being separate modular components thereby allowing the interchangeability described above. Generally, the support strap 14 of the present invention is formed from a flexible polymer material having a low coefficient of elongation such that the flexibility of the support strap 14 allows it to conform to the support surfaces, such as the wearer's shoulder 26 and neck without compression of the width 40 or cross sectional area 42 of the support strap 14.

As was stated above, prior art straps were typically formed from webbing and sometimes included a foam pad arrangement of some kind. Over time the prior art straps compressed in both their thickness and bunched across their widths causing a reduction of the contact area on the wearer's neck and shoulder across which the supported weight was distributed. This bunching caused discomfort in the wearer's shoulders and made it easier for the strap to rid up against and chafe or pinch the wearer's neck. In contrast, as can be seen best in FIG. 5, the support strap 14 of the present invention has a width 40 and a cross sectional area 42 such that the support strap 14 body is formed entirely a flexible polymer material across its entire cross sectional profile. In this regard the cross sectional profile of the support strap 14 is homogeneous and formed only form a single material. This flexible polymer material has sufficient durometer that it will not bunch and when loaded will not experience any significant compression across the width 40 or cross sectional area 42 of the support strap 14 while remaining flexible enough to conform to the contours of the wearer's shoulder and neck. This feature makes the support strap 14 of the present invention much more comfortable for the wearer as compared to the slings in the prior art. Further, the polymer material selected for the support strap 14 of the present invention must have a low coefficient of elongation such that the support strap 14 will not experience any significant stretching along its length when loaded and supporting an average human arm 16 weighing between approximately 11 and 14 pounds.

The support strap 14 of the present invention, being formed from a flexile polymer such as an elastomer or a natural rubber, can be shaped using any known technique for forming polymers including but not limited to extrusion, compression molding and injection molding. In forming the support strap 14, at least a portion of an inner surface thereof is formed to include integrally molded protrusions 44 that serve to better grip the supporting surfaces of the wearer and maintain the support strap 14 in the correct position on the wearer's shoulder and/or neck. These protrusions 44 may be raised bumps, circles, diamonds, pyramids, wavy lines, etc. provided they serve to enhance the gripping action of the contact surface on the inner side of the support strap 14. It should be further appreciated that rather than forming the entire surface of the support strap 14 to include the raised protrusions 44, the support strap 14 may be formed to include a first end 22, a second end 24 and a central contact region 25 that includes the raised protrusions 44. In this regard, as depicted in FIG. 4, the first 22 and second 24 ends and the central region 25 may be stitched, adhered or heat welded together to form the support strap 14.

While the support strap 14 may be formed to have a fixed length using s single piece of material extending from end to end with stitching therein as depicted in FIG. 3, the support strap 14 may also be formed to have an adjustable length as depicted in FIG. 4. The support strap 14 in FIG. 4 can be seen to include one fixed end 24 and an adjustable end 22 that is looped and attached back onto itself using hook and loop fasteners 46. This arrangement allows the support strap 14 to have an adjustable length. In the alternative, buttons, hooks, snaps, etc. may be used in place of the hook and loop fasteners 46 in order to facilitate the adjustment feature of the support strap 44. Such looped ends with releasable fasteners also allows for the support strap 14 to be modular in that it can be easily removed from one sling pouch 12 and affixed to an alternate sling pouch 12.

It can therefore be seen that the present inventing provides a sling assembly 10 that includes an enhanced support strap 14 that better distributes the supported weight and prevents slippage of the support strap 14 relative to the user. Further, the support strap 14 is formed from a flexible material that prevents bunching and eliminates the force concentrations that result therefrom making the support strap 14 and overall sling assembly 10 much more comfortable to wear. Finally, the present invention provides a sling pouch 12 that includes convenient storage areas 30, 34 and has a fashionable flair. In combination, the elements of the present invention serve to provide a sling assembly 10 that will be desirable to the wearer, thereby increasing the level of compliance on the part of the wearer. For these reasons, the instant invention is believed to represent a significant advancement in the art, which has substantial commercial merit.

While there is shown and described herein certain specific structure embodying the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed:

1. A support strap for suspending a sling pouch from a support surface on a wearer's body, said sling pouch including first and second attachment points and being configured for receiving and supporting a wearer's arm, said support strap comprising:
a first end configured for engagement with said first attachment point on said sling pouch;
a second end configured for engagement with said second attachment point on said sling pouch; and
a strap body having a width, a cross sectional area and a length extending between said first and second ends, said strap body being entirely a flexible polymer material having a low coefficient of elongation, said strap conforming to said support surfaces without compression of said width or cross sectional area of said strap.

2. The support strap of claim 1, wherein said strap body is formed using a method selected from the group consisting of: extrusion, compression molding and injection molding.

3. The support strap of claim 1, said strap body further comprising:
a contact surface including integrally formed protrusions extending therefrom to prevent said strap from slipping relative to said support surface.

4. The support strap of claim 3, said strap being formed from three conjoined pieces, a first section including said first end, a second section including said second end and central section extending between said first and second sections and including said contact surface formed thereon.

5. The support strap of claim 1, wherein the length of said strap is selectively adjustable.

6. The support strap of claim 1, wherein said first and second ends of said strap are stitched to said first and second attachment points.

7. The support strap of claim 1, wherein said first and second ends of said strap are releasably engaged with said first and second attachment points.

8. The support strap of claim 1, wherein said cross sectional area of said strap is fully homogeneous and formed from only a single material.

9. The support strap of claim 1, wherein said strap can support weight from said arm contained within said sling substantially without dimensional change.

10. A sling for supporting a wearer's arm from a support surface, said sling comprising:
a sling pouch including first and second attachment points and being configured for receiving and supporting said wearer's arm; and
a support strap including:
a first end configured for engagement with said first attachment point on said sling pouch;
a second end configured for engagement with said second attachment point on said sling pouch; and
a strap body having a width, a cross sectional area and a length extending between said first and second ends, said strap body being entirely a flexible polymer material having a low coefficient of elongation, said strap conforming to said support surfaces without compression of said width or cross sectional area of said strap.

11. The sling of claim 10, wherein said strap body is formed using a method selected from the group consisting of: extrusion, compression molding and injection molding.

12. The sling of claim 10 said strap body further comprising:
a contact surface including integrally formed protrusions extending therefrom to prevent said strap from slipping relative to said support surface.

13. The sling of claim 12, said strap being formed from three conjoined pieces, a first section including said first end, a second section including said second end and central section extending between said first and second sections and including said contact surface formed thereon.

14. The sling of claim 10 wherein the length of said strap is selectively adjustable.

15. The sling of claim 10, wherein said first and second ends of said strap are stitched to said first and second attachment points.

16. The sling of claim 10, wherein said first and second ends of said strap are releasably engaged with said first and second attachment points.

17. The sling of claim 10 wherein said cross sectional area of said strap is fully homogeneous and formed from only a single material.

18. The sling of claim 10, wherein said strap can support weight from an arm contained within said sling substantially without dimensional change.

19. The sling of claim 10, wherein said sling pouch includes a storage pocket for containing said wearer's personal belongings.

20. The sling of claim 10, wherein said sling pouch is formed from a material bearing a pattern thereon said pattern being selected by said wearer.
